(19) United States
(12) Patent Application Publication
(10) Pub. No.: US 2016/0058930 A1
(43) Pub. Date: Mar. 3, 2016

(54) BLOOD PUMP AND METHOD OF SUCTION DETECTION

(71) Applicant: Thoratec Corporation, Pleasanton, CA (US)

(72) Inventors: Alexander Medvedev, Ann Arbor, MI (US); J. Bradford Rainier, Chelsea, MI (US); Muhammad K. Sami, Ypsilanti, MI (US)

(21) Appl. No.: 14/834,771

(22) Filed: Aug. 25, 2015

Related U.S. Application Data
(60) Provisional application No. 62/041,917, filed on Aug. 26, 2014.

(51) Int. Cl. A61M 1/10

(52) U.S. Cl. CPC A61M 1/1086; A61M 2205/3334; A61M 2205/3365; A61M 2205/50

(57) ABSTRACT

A system and method for detecting and mitigating a suction condition are disclosed. The method may include characterizing pump waveform signal, identifying and evaluating a characteristic of the waveform for an existence of a suction condition. In various embodiments, a change in harmonic spectral distribution will identify a probability of a suction condition. A speed of the pump may be adjusted to mitigate the suction condition.

### CROSS-REFERENCE TO RELATED APPLICATIONS

This application claims priority to Provisional U.S. Patent Application No. 62/041,917 filed Aug. 26, 2014, entitled “BLOOD PUMP AND METHOD OF SUCTION DETECTION,” the entire disclosure of which is hereby incorporated by reference, for all purposes, as if fully set forth herein.

### BACKGROUND OF THE INVENTION

This invention relates, in general, to mechanical circulatory support systems and methods for their use. Various aspects of the invention relate to methods of detecting and mitigating ventricular suction events.

Suction detection and prevention is critical for heart failure patients supported by blood pumps (e.g. a ventricular assist device or VAD). In the case of a VAD, a suction event refers to an instance of negative pressure created in the ventricle. A suction event, which is typically triggered by a pump speed too high for the given systemic conditions and a patient's physiology, affects clinical outcomes and can lead to major adverse events in extreme cases. Suction events can be mitigated by lowering the pump speed when such an event is detected. A speed reduction may not completely eliminate suction, but it will reduce the likelihood of a continuous severe suction condition under normal pump operation.

One typical method for detecting a suction event includes the trend analysis of a pulsatility index (PI). PI is a measure of the variability of blood velocity in a vessel, and in the case of a VAD, PI is a measure of the pressure differential inside the VAD pump during the native heart's cardiac cycle and represents volume status, right ventricle function, and native heart contractility. PI may be calculated taking into consideration factors such as pump power, current, back electromotive force (emf). Another method for detecting a suction event includes correlating the pump flow waveform to a database of signals indicating suction events. Yet another method includes performing a harmonic analysis of the pump power or pump flow waveform. Exemplars of existing suction detection techniques are described in U.S. Pat. No. 7,645,225 to Medvedev, U.S. Pat. No. 7,175,588 to Morello, U.S. Pat. No. 6,991,595 to Burke et al., and U.S. Pat. No. 5,888,242 to Antaki et al. and U.S. Pub. No. 2014/0100413 to Casas, which are incorporated herein for all purposes by reference.

Existing methods for detecting the imminence or presence of a suction event have several limitations. Methods other than waveform correlation are limited in their capability to discern a suction event when compared to other patient physiological conditions that may not have any relevance to a suction condition (U.S. Pub. No. 2014/0100413 to Casas). Accordingly, the results can be inaccurate and lead to false positive detection of a suction event. Although waveform correlation methods can be more accurate, these techniques are challenging to implement because they require a database of suction event signals against the input signal be matched (U.S. Pat. No. 7,175,588 to Morello). The correlation of signals also requires extensive signal processing capabilities. Such capabilities are typically not available in embedded systems used to drive LVAD pumps. Extensive signal processing also tends to lead to greater energy usage and heat which can be challenging when the components are implanted in the body or directly against the skin.

What is needed are devices and methods which overcome the above disadvantages. What is needed is an improved suction detection technique.

### BRIEF SUMMARY OF THE INVENTION

Various aspects of the invention are directed to a method of detecting a suction event of a blood pump by obtaining a flow waveform of the pump and evaluating a characteristic of the waveform for an existence of a suction condition. In various embodiments, the evaluation comprises evaluating Harmonic Spectral Distribution (HSD) to identify a probability of a suction condition.

One exemplary method of detecting a suction event in a blood pump comprises determining at least one operating parameter of a blood pump. Based on the operating parameter of the blood pump, a characteristic waveform signal of the blood pump is calculated. A suction index of the characteristic waveform signal is calculated and evaluated to see if the suction index exceeds a predefined threshold. The occurance of a suction event is determined when the suction index exceeds the predefined threshold. In some cases, the operation of the pump may be altered when such a suction event is determined. This may be accomplished, for example, by configuring a computer controller to change the operation of the blood pump after the controller detects the suction event.

In one aspect, the operating parameter may comprise one or more of the following: pump power, pump voltage, pump current, pump speed, or the like. In another aspect, the characteristic waveform signal comprises a representation of flow per time, current per time or power per time of the blood pump.

In some cases, the suction index may be calculated based on a Harmonic Spectral Distribution by executing a Fast Hartley Transform on the characteristic waveform signal. As one example, the Harmonic Spectral Distribution may be calculated by executing a Discrete Bracewell Transform to evaluate the Fast Hartley Transform.

In one aspect, the suction condition exists when the suction index is greater than 20%. In another aspect, a speed of the blood pump may be lowered using a binary search method in response to a determination that the suction event has occurred. As an example, the speed of the pump may be lowered to a speed which is based at least in part on the Harmonic Spectral Distribution and a low pump speed limit. In some cases, the speed of the blood pump may be increased using a binary search method until a set-point speed is restored.

In yet another aspect, a determination may be made to see if the characteristic waveform signal is valid based at least in part on at least one pump performance characteristic of the blood pump. A notification is provided when the characteristic waveform signal is not valid.

The invention may further include a system for detecting the occurrence of a suction event of a blood pump using the techniques described herein. One or more computer systems or computer controllers may also be used to control operation of a pump based on the detection of such a suction event.

The systems and methods of the present invention have other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

FIG. 1 is a schematic of a system for controlling a blood pump in accordance with the present invention.

FIG. 2A is a flow diagram illustrating a method for detecting and mitigating a suction event.

FIG. 2B is a block diagram of the components of an exemplary system for implementing the method of FIG. 2A.

FIG. 3 is a line chart showing an input flow waveform signal and its corresponding harmonic spectral distribution for evaluating the probability of a suction condition in accordance with the invention.

FIG. 4 is a photo illustrating an experimental mock loop representation of the system of FIG. 1.

FIG. 5 shows an example computing system or device.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

For example, any detail discussed with regard to one embodiment may or may not be present in all contemplated versions of that embodiment. Likewise, any detail discussed with regard to one embodiment may or may not be present in all contemplated versions of other embodiments discussed herein. Finally, the absence of discussion of any detail with regard to embodiment herein shall be an implicit recognition that such detail may or may not be present in any version of any embodiment discussed herein.

Specific details are given in the following description to provide a thorough understanding of the embodiments. However, it will be understood by one of ordinary skill in the art that the embodiments may be practiced without these specific details. For example, circuits, systems, networks, processes, and other elements in the invention may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

The term “machine-readable medium” includes, but is not limited to transitory and non-transitory, portable or fixed storage devices, optical storage devices, wireless channels and various other mediums capable of storing, containing or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments of the invention may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

For convenience in explanation and accurate definition in the appended claims, the terms “up” or “upper”, “down” or “lower”, “inside” and “outside” are used to describe features of the present invention with reference to the positions of such features as displayed in the figures.

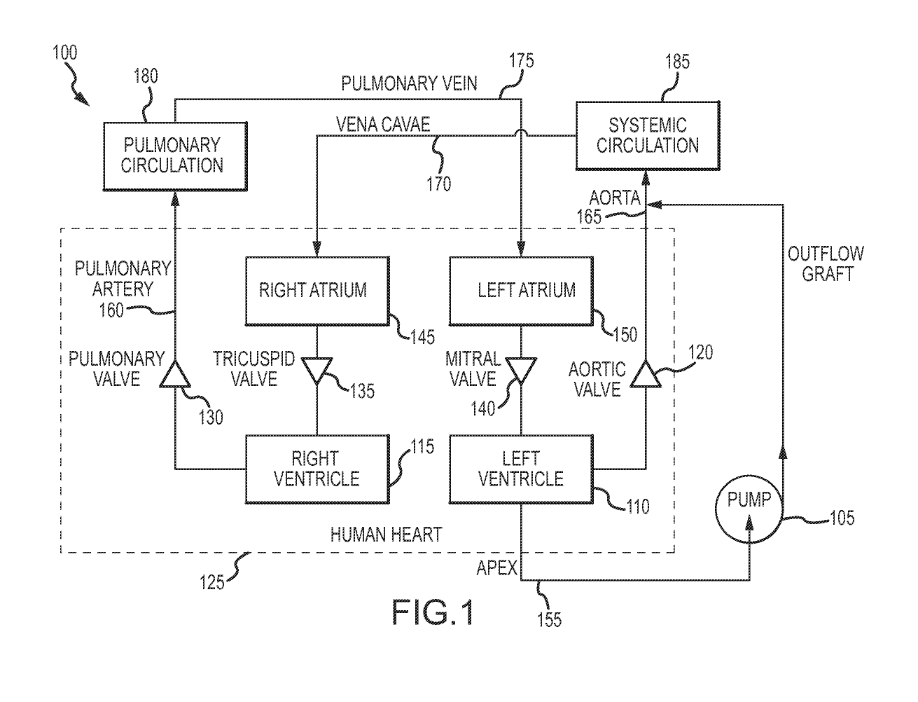

Turning now to the drawings, wherein like components are designated by like reference numerals throughout the various figures, attention is directed to FIGS. 1 to 4. Various aspects are similar to those described in and U.S. Pub. Nos. 2014/0100413 to Casas, 2011/0129373 to Mori, 2014/0155682 to Jeffery et al., and 2013/0225909 to Dormanen et al., and U.S. Pat. No. 7,850,594 to Sutton, U.S. Pat. No. 7,645,225 to Medvedev, U.S. Pat. No. 7,175,588 to Morello, U.S. Pat. No. 6,991,595 to Burke et al., U.S. Pat. No. 5,888,242 to Antaki et al., and U.S. Pat. No. 6,066,086 to Antaki et al., the entire contents of which patents and applications are incorporated herein for all purposes by reference.

As shown in FIG. 1, in various embodiments, the basic design concept 100 includes a pump 105 connected to the left ventricle 110 (or right ventricle 115 or both ventricles) across the aortic valve 120 of a heart 125. The flow through pump 105 is estimated using pump parameters which include power/current, speed and a flow map, which allows transformation of inputs like power/current and speed to flow based on the pump flow characteristics curves (e.g. power/current-flow curves for various speeds and viscosities/hematocrits). Heart 125 also includes pulmonary valve 130, tricuspid valve 135, mitral valve 140, right atrium 145, and left atrium 150. Apex 155, pulmonary artery 160, aorta 165, vena cavae 170, and pulmonary vein 175 are also shown in FIG. 1. Blocks representing pulmonary circulation 180 and systemic circulation 185 are also shown. The flow can also be obtained by a direct measurement using various flow probes.

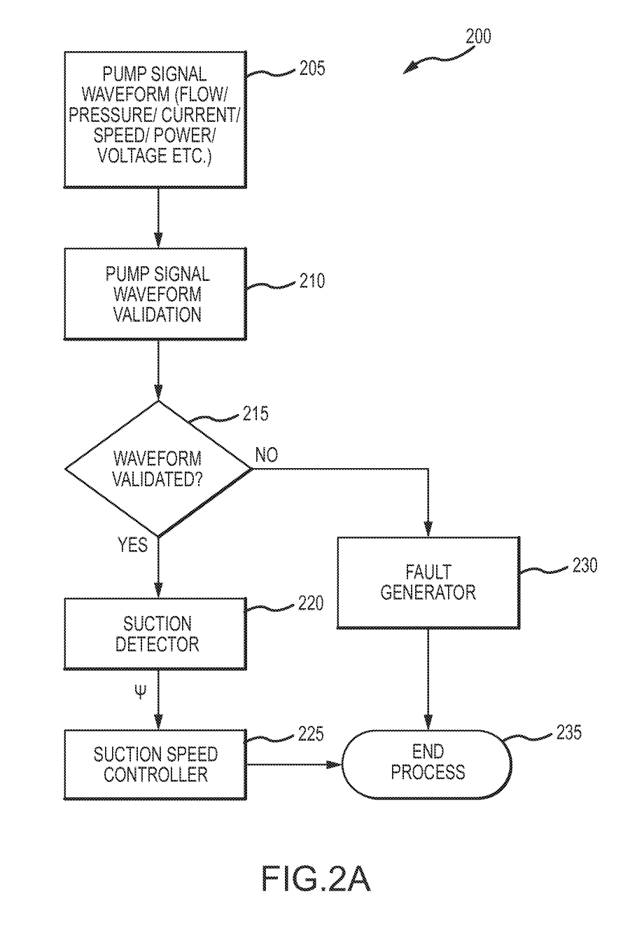

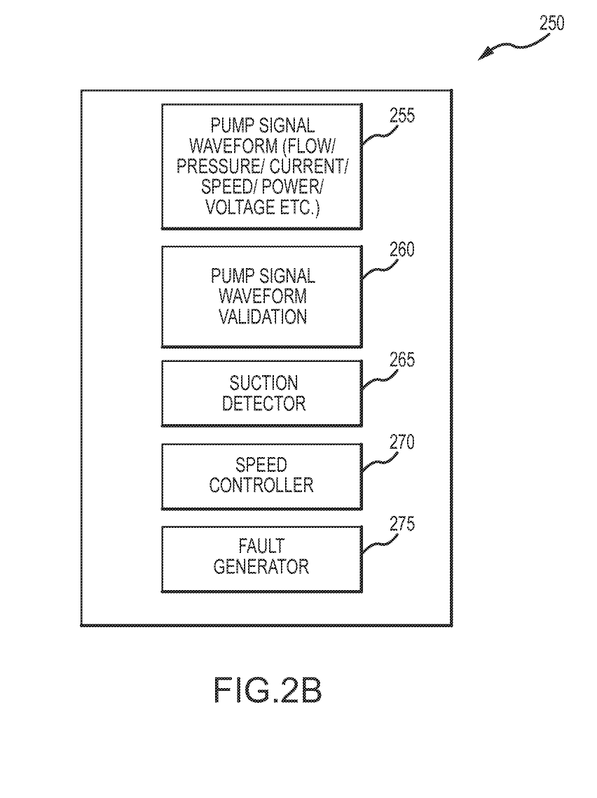

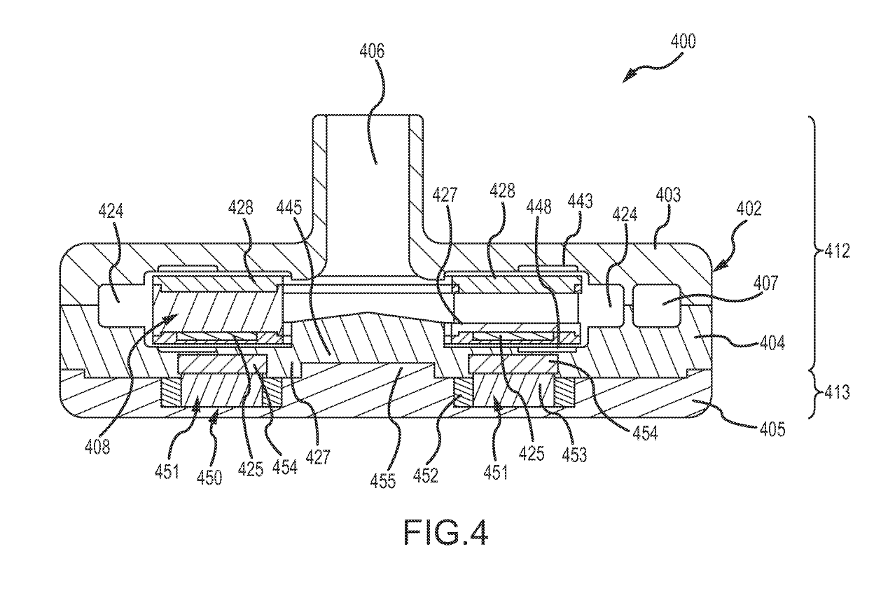

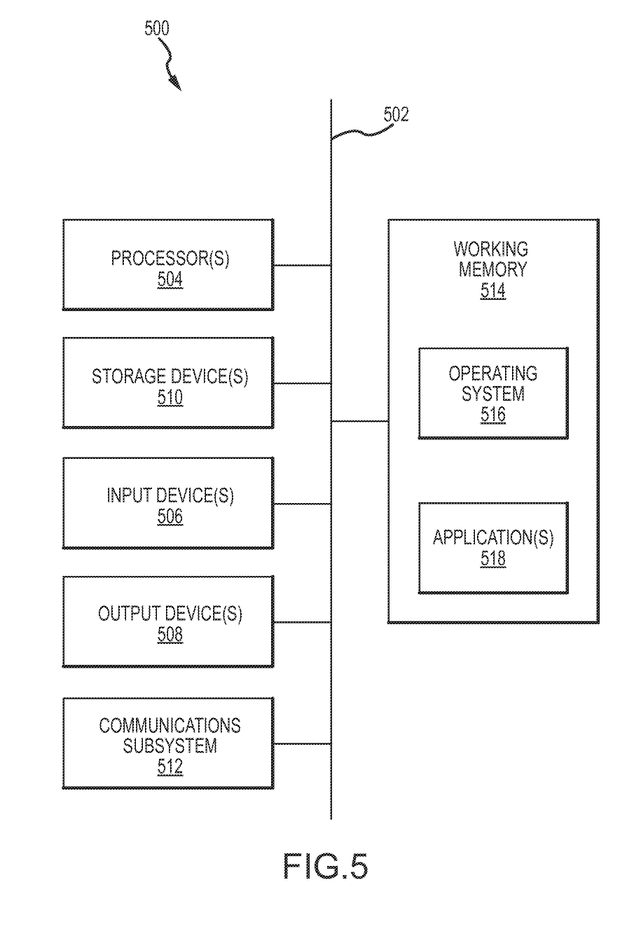

FIG. 2A illustrates the flow of a method 200 in accordance with the inventions. FIG. 2B illustrates the components of an exemplary physiological control system 250 implementing the suction detection technique in accordance with the inventions. It is contemplated that the components shown in FIG. 2B may be implemented by at least one computing system or device in or as software, firmware, or hardware, and/or any combination thereof. An example of such a computing system or device is shown and described in connection with FIG. 5. Example functions for suction detection and mitigation using the illustrated system will now be described with respect to an exemplary left ventricular assist device (LVAD) (shown in FIG. 4).

Referring to FIGS. 2A and 2B, in block 205 the Pump Signal Waveform 255 takes various pump and system parameters like pump speed, pump current, pump voltage, pump power transfer characteristics and other system or vital-related parameters like Hematocrit to provide the characteristic pump signal waveform. The pump characteristics can be used to identify and validate the pump signal waveform.

Referring to FIGS. 2A and 2B, in block 210 a Pump Signal Waveform Validation 260 takes the Pump Signal and validates it based on pump performance characteristics to make a determination at block 215.

If the waveform is validated, then at block 220, a Suction Detector 265 evaluates the existence of the suction condition using the method described below. For the exemplary application, Ψ>20% is used as a determining factor for the declaration of the presence of suction.

In block 225, the value from the Suction Detector 30 is passed on to a Suction Speed Controller 270 to make appropriate adjustments to the speed according the pulse suction index Ψ output.

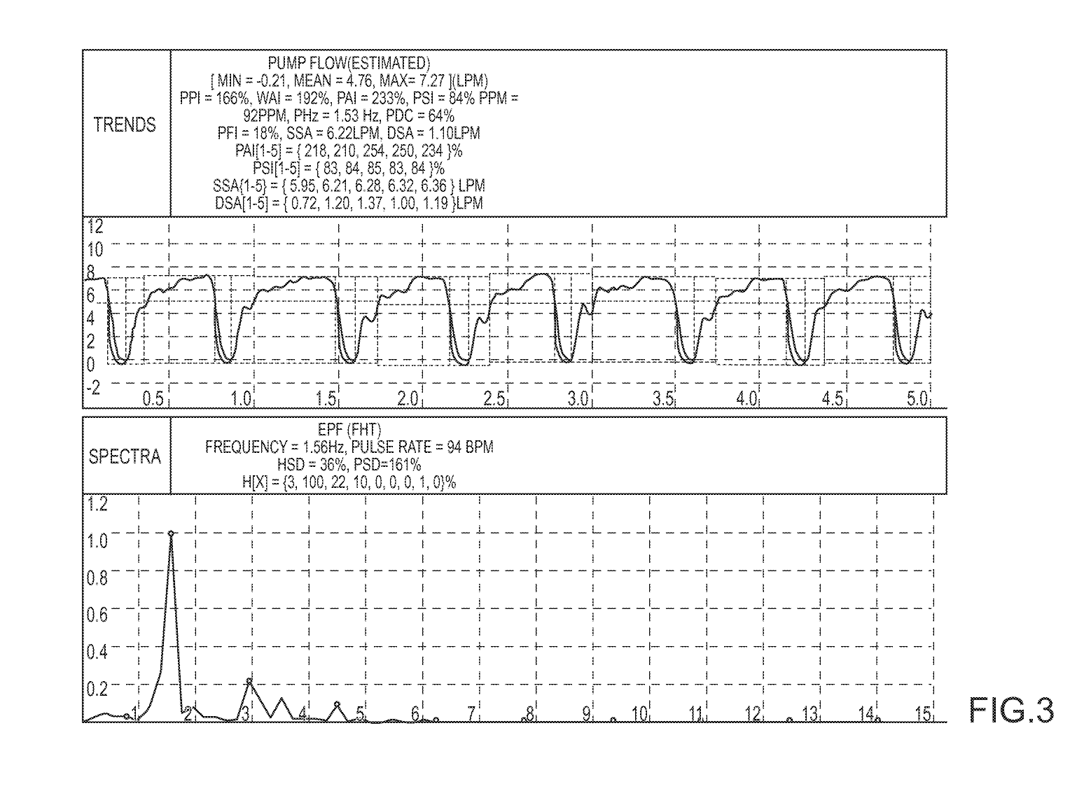

The index PSI/SI/Ψ is calculated by using the Harmonic Spectral Distribution (HSD) of the pump flow waveform as shown in the lowermost plot of FIG. 3. The waveform could be derived from any of the pump power, pump current, pump head pressure, speed, etc.

With reference to FIG. 2A and block 220, the harmonic spectral distribution (HSD) is calculated using the Fast Hartley Transform (FHT). To avoid spurious suction events mean value for consecutive HSD can be used or the HSD can be filtered using a low pass filter.

With continued reference to FIGS. 2A and 2B, in block 225 Suction Speed Controller 270 takes the suction index Ψ and low pump speed limit as inputs, and based on current pump speed and historical presence of suction condition, makes adjustments to the pump speed necessary to mitigate a suction condition or to recover the clinician specified patient speed while minimizing the introduction of suction events over time as the speed is restored to its set-point. For both speed reduction and speed increase a binary search strategy may be used for its fast convergence to the optimal speed set-point along with a maximum upper bound for the speed set-point step change.

While this type of control provides a means of suction mitigation, in some clinical settings and for some patient physiologies a means to disable the control of speed will be required. For those scenarios a provision to enable/disable Suction Speed Control may be desirable as a user configurable item. The default value may be enabled, and the feature can be disabled by the clinician as needed.

In block 230, a fault generator 275 is intended to provide visual/audible notifications to the patient when pump signal validation fails. The fault generator may also provide a notification when a suction condition is detected that needs mitigation. These conditions may include low/no mean flow, low peak-to-peak flow, pulse rate too high or too low, suction detected, unmitigated suction and inflow/outflow obstruction conditions. The determination and selection of faults to the patient is a risk management activity. The criteria for notification to the patient may be pre-selected by a risk management team.

In block 235, the physiological control process ends.

The methods and systems described herein for suction detection and mitigation provides several advantages over existing techniques. As described herein, the mitigation in response to the presence of a suction condition is optimized for its response time and minimizes the need for clinician involvement compared to existing approaches. It also minimizes the number of necessary patient visits in cases where there is a recovery from patient's physiological conditions causing suction condition over time.

FIG. 4 shows a schematic of a blood pump 400 in accordance with some embodiments of the invention. The techniques of the invention may be used in connection with a blood pump of this type, among others. As such, it will be appreciated that the invention is not intended to be limited to a specific embodiment of a blood pump but may be used with a wide variety of such pumps. Blood pump 400 includes a housing 402 having a blood inlet port 406 and a blood outlet port (not shown) via conduit 407; a pump unit 412 including an impeller 408 which has a plurality of magnetic materials (magnetic material bodies or pieces) 425 and which rotates within the housing to feed blood; and an impeller rotational torque generation section 413 for rotating the impeller. Housing 402 includes a plurality of magnetic members 454 embedded between the impeller 408 and the impeller rotational torque generation section 413 for transmitting a magnetically attractive force generated by the impeller rotational torque generation section 413 to the magnetic material bodies 425 of the impeller. The magnetic material bodies 454 are embedded in the housing 402 (second housing member 404) so that the magnetic material bodies 454 are positioned in respective recesses in the housing 402 (second housing member 404) and so that the magnetic material bodies 454 form a part of the housing 402 or second housing member 404 (e.g., the material forming the second housing member 404 contacts and surrounds at least a portion of the magnetic material bodies 454 as shown in FIG. 4). The blood pump 400 includes a non-contact bearing mechanism for rotating the impeller without contacting within the housing when the impeller is rotated by the impeller rotational torque generation section 413.

The blood pump apparatus 400 in the present embodiment includes the housing 402, the pump unit 412 composed of the impeller 408 accommodated in the housing 402, and the impeller rotational torque generation section 413 for rotating the impeller. In addition, in the blood pump apparatus 400 in the present embodiment, the impeller rotational torque generation section 413 is attachable to and detachable from the pump unit 412. With the impeller rotational torque generation section 413 thus attachable to and detachable from the pump unit 412, the impeller rotational torque generation section 413 having no blood contact part during use can be reused, so that only the pump unit 412 which has a blood circulating part is disposable.

The housing 402 includes: a first housing member 403 having the blood inlet port 406 and a recess for accommodating an upper portion of the impeller 408; and a second housing member 404 having the blood outlet port and a recess for accommodating a lower portion of the impeller 408. The housing 402 is formed by combining the first housing member 403 and the second housing member 404 with each other. The interior of the housing 402 is provided with or forms a blood chamber 424 through which the blood inlet port 406 and the blood outlet port communicate with each other. The blood inlet port 406 projects substantially perpendicularly from around the center of the upper surface of the housing 402 (the first housing member 403). The blood inlet port 406 is not limited to the straight pipe as illustrated, but may be a curved pipe or a bent pipe. The blood outlet port projects in a tangential direction from the side surface of the housing 402, which is formed in a substantially hollow cylindrical shape. According to this disclosed embodiment, the blood outflow passage is of a double volute structure divided into two parts in the, but it may be of a single volute structure or of a voluteless structure.

The housing 402 includes the plurality of magnetic members 454 embedded between the impeller 408 and the impeller rotational torque generation section 413 for transmitting a magnetically attractive force generated by the impeller rotational torque generation section 413 to the magnetic material bodies 425 of the impeller. Specifically, the plurality of magnetic members 454 are embedded in the second housing member 404 (more specifically, in the bottom wall of the second housing member 404). It is particularly preferable that the magnetic members 454 are so embedded as not to be exposed to the inside of the blood chamber 424, as in the pump apparatus 1 according to the present embodiment. As the magnetic member 454, a ferromagnetic material is used.

The housing 402, specifically the first housing member 403 and the second housing member 404, are formed of synthetic resin or metal. In addition, the first housing member 403 and the second housing member 404 have peripheral parts which make surface contact with each other, as shown in FIG. 4.

The impeller 408 is contained in the housing 402. Specifically, as shown in FIG. 4, a disk-shaped impeller 408 provided with a centrally located through-hole is contained in the blood chamber 424 formed inside the housing 402.

As shown in FIG. 4, the impeller 408 includes an annular member (lower shroud) 427 forming a lower surface, an annular member (upper shroud) 428 provided with an opening in its center and forming an upper surface, and a plurality of (for example, seven) vanes between the two members or shrouds. Between the lower shroud and the upper shroud, there are formed a plurality of (for example, seven) blood flow channels, each partitioned by the adjacent vanes. The blood flow channels communicate with the central opening of the impeller 408, and extend to the outer peripheral edge while gradually increasing in width, starting from the central opening of the impeller 408. In other words, the vanes are each formed between the adjacent blood flow channels. In the present embodiment, the blood flow channels and the vanes are provided at regular angular intervals and in substantially the same shape, respectively.

As shown in FIG. 4, the impeller 408 has a plurality of (for example, six) magnetic material bodies or pieces 425 (permanent magnets; driven magnets) embedded therein. In the present embodiment, the magnetic material bodies 425 are embedded in the lower shroud 427. The magnetic material bodies 425 (permanent magnets) thus embedded are attracted toward the impeller rotational torque generation section 413 side by stators 451 of the impeller rotational torque generation section 413 and, also, receive a rotation torque of the impeller rotational torque generation section 413 through the magnetic members embedded in the housing 402 (the second housing member 404).

In addition, where a certain number of magnetic bodies 425 are embedded as in the present embodiment, magnetic coupling with the plurality of stators 451 can be secured sufficiently. Preferred shapes of the magnetic material bodies 425 (permanent magnet) include a circle, a sector and, further, a ring (an integral form in which N poles and S poles are alternately polarized). The impeller members are formed of a highly corrosion-resistant metal (titanium, stainless steel SUS316L, or the like) or synthetic resin. As the synthetic resin here, those which have been described above as material for the housing can be preferably used.

The blood pump apparatus 400 disclosed here includes a non-contact bearing mechanism for rotating the impeller without contacting the inner surface of the housing when the impeller is rotated by the impeller rotational torque generation section 413.

In the pump apparatus 400 disclosed here, the non-contact bearing mechanism is composed of grooves for hydrodynamic bearing 448 provided in the inner surface of the housing 402 on the impeller rotational torque generation section 413 side, in other words in a surface (bottom wall surface) of the recess in the second housing member 404. The impeller is rotated, without contact, under a dynamic pressure bearing effect offered by a dynamic pressure generated between a surface (groove for hydrodynamic bearing formed part) 442 in which the grooves for hydrodynamic bearing are formed and the impeller 408, by rotation thereof at a rotating speed of not less than a predetermined value. The groove for hydrodynamic bearing formed part is formed in a size corresponding to a bottom surface (a surface on the impeller rotational torque generation section side) of the impeller 408. In the pump apparatus 400 disclosed here, each of the grooves for hydrodynamic bearing 448 has its one end on the peripheral edge (circumference) of a circular part slightly outwardly spaced from the center of the surface of the recess in the second housing member, and extends therefrom nearly to the outer edge of the recess surface in a vortex form (in other words, in a curved form) while gradually increasing in width. The grooves for hydrodynamic bearing 448 are plural in number, are the same shape (inclusive of substantially the same shape), and are arranged at regular (equal) intervals (inclusive of substantially equal intervals). The grooves for hydrodynamic bearing 448 are each a recess, the depth of which is preferably about 0.005 to 0.4 mm. The number of the grooves for hydrodynamic bearing 448 is preferably about 6 to 36. In the present example, twelve grooves for hydrodynamic bearing are arranged at regular (equal) angular intervals about the center axis of the impeller. The grooves for hydrodynamic bearing 448 in the pump apparatus disclosed here have a so-called inward spiral groove shape. In the process of pumping fluid by the action of the groove for hydrodynamic bearing formed part, clockwise rotation of the impeller raises the pressure from the outer diameter side toward the inner diameter side of the groove part, so that a force in the opposite direction is obtained between the impeller 408 and the housing 402 forming the groove for hydrodynamic bearing formed part, and this force serves as a dynamic pressure.

The impeller 408 is attracted toward the impeller rotational torque generation section 413 side at the time of rotation. The presence of the groove for hydrodynamic bearing formed part as above-mentioned helps ensure that, by the dynamic pressure bearing effect provided between the groove for hydrodynamic bearing formed part of the housing and the bottom surface of the impeller 408 (or between the groove for hydrodynamic bearing formed part of the impeller and the housing inner surface), the impeller 408 is separated from the housing inner surface, and is rotated without contact, whereby a blood flow channel is secured between the lower surface of the impeller and the housing inner surface, and blood stagnation between these surfaces and the resultant thrombus formation are prevented from occurring. Further, in a normal condition, the groove for hydrodynamic bearing formed part exhibits a stirring action between the lower surface of the impeller and the housing inner surface, so that partial blood stagnation between these surfaces is inhibited or prevented from occurring.

The groove for hydrodynamic bearing formed part may be provided in that surface of the impeller 408 which is on the impeller rotational torque generation section side, not on the housing side. In this case, also, the same configuration as that of the groove for hydrodynamic bearing formed part described above is preferably adopted. Specifically, the grooves for hydrodynamic bearing may be provided in that surface of the impeller 408 which is on the impeller rotational torque generation section 413 side (in other words, in the bottom surface of the impeller 408).

The pump apparatus 400 in the present embodiment can be constructed so that the housing inner surface on the opposite side to the impeller rotational torque generating part side (i.e., the surface of the recess in the first housing member 403) may also be provided with a groove for hydrodynamic bearing formed part (second groove for hydrodynamic bearing formed part) having a plurality of grooves for hydrodynamic bearing (second grooves for hydrodynamic bearing) 433.

The impeller rotational torque generation section 413 of the blood pump apparatus 400 according to the present embodiment, as shown in FIG. 4, is composed of a motor stator 50 including a plurality of stators 451 disposed on the circumference of a circle (arranged in an annular form). A third housing member 405 is provided with an annular recess (doughnut-shaped recess), and the plurality of stators 451 are contained in the third housing member 405, in the state of being arranged in an annular pattern (doughnut-like pattern). The stator 451 has a stator core 453 and a stator coil 452 wound around the stator core 453. In the pump apparatus 400 according to the present embodiment, six stators 451 form the stator motor 450. As the stator coil 452, a multilayer wound stator coil is used. With the direction of current flowing in the stator coils 452 of the respective stators 451 switched over or alternating a rotating magnetic field is generated, by which the impeller is attracted and rotated.

In the blood pump apparatus 400 in the present embodiment, as shown in FIG. 4, the respective magnetic members 454 of the housing 402 (specifically, the second housing member 404) are so disposed as to be located on, or in overlying relation to, the stator cores 453 of the respective stators 451 described above. That is, each of the plurality of magnetic members 454 is positioned in circumferential alignment with one of the stator cores 453 of the stators 451. The stator cores 453 in the present embodiment are each sector-shaped a, and correspondingly, the magnetic members 454 are also each sector-shaped. The magnetic members 454 are slightly greater in size than the stator cores 453.

Further, in the blood pump apparatus 400 according to the present embodiment, as shown in FIG. 4, each of the magnetic members 454 of the housing 402 (specifically, the second housing member 404) makes direct contact with the stator core 453 of each of the stators 451. More specifically, in this pump apparatus 400, an upper end portion of the stator core 453 projects upwardly slightly beyond the stator coil 452, and the projecting portion is exposed. The magnetic member 454 is so embedded in the second housing member 404 that its lower surface is exposed; further, the portion where the lower surface of the magnetic member 454 is exposed forms a recess in which the projecting portion of the stator core 453 is accommodated. Therefore, the magnetic member 454 and the stator core 453 are in contact with each other. This helps ensure that a magnetic force generated in the stator 451 can be securely transmitted to the magnetic member 454.

In the pump apparatus 400 according to the present embodiment, the pump unit 412 and the impeller rotational torque generation section 413 can be attached to and detached from each other, and both of them have a connecting mechanism. In the pump apparatus 400 in the present embodiment, the second housing member of the pump unit 412 is provided at its bottom surface with a first engaging part (a recess) 445, whereas the housing 405 of the impeller rotational torque generation section 413 is provided with a second engaging part (specifically, a projection) 455 which engages the first engaging part (recess) 445. The engagement between the first engaging part (recess) 445 of the pump unit 412 and the second engaging part (projection) 455 of the impeller rotational torque generation section 413 connects the units to each other.

FIG. 5 shows an example computer system or device 500 in accordance with the disclosure. An example of a computer system or device includes a medical device, a desktop computer, a laptop computer, a tablet computer, and/or any other type of machine configured for performing calculations.

The example computer device 500 may be configured to perform and/or include instructions that, when executed, cause the computer system 500 to perform a method for or of detecting a suction event of a blood pump such as that discussed in the context of the present disclosure. For example, the example computer device 500 may be configured to perform and/or include instructions that, when executed, cause the computer system 500 to perform at least one of the following steps: determining a characteristic waveform signal of the pump; identifying the transients in the waveform based on the obtained pump signal; and evaluating a characteristic of the waveform for an existence of a suction condition.

Additionally, or alternatively, the example computer device 500 may be configured to perform and/or include instructions that, when executed, cause the computer system 500 to determine a quantity of the suction condition, where the quantity is named after the Greek Symbol (Ψ) pronounced as PSI and used as an acronym with the definition of Pulse Suction Index (PSI).

Additionally, or alternatively, the example computer device 500 may be configured to perform and/or include instructions that, when executed, cause the computer system 500 to compare PSI with a predetermined threshold to identify suction.

Additionally, or alternatively, the example computer device 500 may be configured to perform and/or include instructions that, when executed, cause the computer system 500 to provide an indication wherein a decrease in PSI(Ψ) with a decrease in speed is used to indicate a likely recovery from suction condition.

Additionally, or alternatively, the example computer device 500 may be configured to perform and/or include instructions that, when executed, cause the computer system 500 to use a binary search to increase or decrease the pump speed when suction criteria is met, and the step change of the pump speed of the binary search is bounded by an upper limit identified as safe for the patient population.

Additionally, or alternatively, the example computer device 500 may be configured to perform and/or include instructions that, when executed, cause the computer system 500 to implement a step wherein suction detection using the analysis of a pump waveform signal, comprises: calculating the Fast Hartley Transform (FHT); calculating Harmonic Spectral Distribution (HSD) for signal analysis;

$$H ( v ) = ( {N}^{- 1} ) {\sum }_{τ = 0}^{N - 1} f ( τ ) cas ( 2 π v τ / N )$$

$$where , \\ v = 0 , 1 , … , N - 1$$

$$cas θ = cos θ + sin θ .$$

Additionally, or alternatively, the example computer device 500 may be configured to perform and/or include instructions that, when executed, cause the computer system 500 to implement a step wherein suction detection using the analysis of the blood pump waveform signal, comprises: calculating an approximation of the Fast Hartley Transform (FHT) using a Discrete Bracewell Transform (DBT); and performing a Harmonic Analysis of the flow waveform; calculating Harmonic Spectral Distribution (HSD) for signal analysis.

Additionally, or alternatively, the example computer device 500 may be configured to perform and/or include instructions that, when executed, cause the computer system 500 to implement a method of or for calculating a pulse suction index (Ψ) as Harmonic Spectral Distribution (HSD):

$$Ψ = HSD = 100 \times \frac{( {\sum }_{x = 2}^{x = n} {H}_{x} + {H}_{1 / 2} )}{{H}_{1}}$$

$$Where , \\ \begin{matrix}{H}_{x} = Value of Harmonic x . \\ {=}^{n} Minimum value is 2 and maximum is from 5 to 9.\end{matrix}$$

Additionally, or alternatively, the example computer device 500 may be configured to perform and/or include instructions that, when executed, cause the computer system 500 to calculate HSD in consecutive time intervals to identify the change in HSD over a period and calculating MEAN value of HSD.

Additionally, or alternatively, the example computer device 500 may be configured to perform and/or include instructions that, when executed, cause the computer system 500 to provide an indication wherein an increase in MEAN value of HSD indicates a suction event.

Additionally, or alternatively, the example computer device 500 may be configured to perform and/or include instructions that, when executed, cause the computer system 500 to provide an indication wherein a decrease in MEAN value of HSD with the decrease in speed may additionally indicate recovery from suction condition.

Additionally, or alternatively, the example computer device 500 may be configured to perform and/or include instructions that, when executed, cause the computer system 500 to identify or determine or detect that a lowest allowable pump speed for recovery from suction condition is based on a Low Speed Limit. In some examples, the Low Speed Limit is based on input from a clinician.

Additionally, or alternatively, the example computer device 500 may be configured to perform and/or include instructions that, when executed, cause the computer system 500 to implement the step of decreasing a speed of the pump in response to identification of the existence of a suction condition.

The computer device 500 is shown comprising hardware elements that may be electrically coupled via a bus 502 (or may otherwise be in communication, as appropriate). The hardware elements may include a processing unit with one or more processors 504, including without limitation one or more general-purpose processors and/or one or more special-purpose processors (such as digital signal processing chips, graphics acceleration processors, and/or the like); one or more input devices 506, which may include without limitation a remote control, a mouse, a keyboard, and/or the like; and one or more output devices 508, which may include without limitation a presentation device (e.g., television), a printer, and/or the like.

The computer system 500 may further include (and/or be in communication with) one or more non-transitory storage devices 510, which may comprise, without limitation, local and/or network accessible storage, and/or may include, without limitation, a disk drive, a drive array, an optical storage device, a solid-state storage device, such as a random access memory, and/or a read-only memory, which may be programmable, flash-updateable, and/or the like. Such storage devices may be configured to implement any appropriate data stores, including without limitation, various file systems, database structures, and/or the like.

The computer device 500 might also include a communications subsystem 512, which may include without limitation a modem, a network card (wireless and/or wired), an infrared communication device, a wireless communication device and/or a chipset such as a Bluetooth™ device, 502.11 device, WiFi device, WiMax device, cellular communication facilities such as GSM (Global System for Mobile Communications), W-CDMA (Wideband Code Division Multiple Access), LTE (Long Term Evolution), etc., and/or the like. The communications subsystem 512 may permit data to be exchanged with a network (such as the network described below, to name one example), other computer systems, and/or any other devices described herein. In many embodiments, the computer system 500 will further comprise a working memory 514, which may include a random access memory and/or a read-only memory device, as described above.

The computer device 500 also may comprise software elements, shown as being currently located within the working memory 514, including an operating system 516, device drivers, executable libraries, and/or other code, such as one or more application programs 518, which may comprise computer programs provided by various embodiments, and/or may be designed to implement methods, and/or configure systems, provided by other embodiments, as described herein. By way of example, one or more procedures described with respect to the method(s) discussed above, and/or system components might be implemented as code and/or instructions executable by a computer (and/or a processor within a computer); in an aspect, then, such code and/or instructions may be used to configure and/or adapt a general purpose computer (or other device) to perform one or more operations in accordance with the described methods.

A set of these instructions and/or code might be stored on a non-transitory computer-readable storage medium, such as the storage device(s) 510 described above. In some cases, the storage medium might be incorporated within a computer system, such as computer system 500. In other embodiments, the storage medium might be separate from a computer system (e.g., a removable medium, such as flash memory), and/or provided in an installation package, such that the storage medium may be used to program, configure, and/or adapt a general purpose computer with the instructions/code stored thereon. These instructions might take the form of executable code, which is executable by the computer device 500 and/or might take the form of source and/or installable code, which, upon compilation and/or installation on the computer system 500 (e.g., using any of a variety of generally available compilers, installation programs, compression/decompression utilities, etc.), then takes the form of executable code.

It will be apparent that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer device 500) to perform methods in accordance with various embodiments of the disclosure. According to a set of embodiments, some or all of the procedures of such methods are performed by the computer system 500 in response to processor 504 executing one or more sequences of one or more instructions (which might be incorporated into the operating system 516 and/or other code, such as an application program 518) contained in the working memory 514. Such instructions may be read into the working memory 514 from another computer-readable medium, such as one or more of the storage device(s) 510. Merely by way of example, execution of the sequences of instructions contained in the working memory 514 may cause the processor(s) 504 to perform one or more procedures of the methods described herein.

The terms “machine-readable medium” and “computer-readable medium,” as used herein, may refer to any non-transitory medium that participates in providing data that causes a machine to operate in a specific fashion. In an embodiment implemented using the computer device 500, various computer-readable media might be involved in providing instructions/code to processor(s) 504 for execution and/or might be used to store and/or carry such instructions/code. In many implementations, a computer-readable medium is a physical and/or tangible storage medium. Such a medium may take the form of a non-volatile media or volatile media. Non-volatile media may include, for example, optical and/or magnetic disks, such as the storage device(s) 510. Volatile media may include, without limitation, dynamic memory, such as the working memory 514.

Example forms of physical and/or tangible computer-readable media may include a floppy disk, a flexible disk, hard disk, magnetic tape, or any other magnetic medium, a compact disc, any other optical medium, ROM (Read Only Memory), RAM (Random Access Memory), and etc., any other memory chip or cartridge, or any other medium from which a computer may read instructions and/or code. Various forms of computer-readable media may be involved in carrying one or more sequences of one or more instructions to the processor(s) 504 for execution. By way of example, the instructions may initially be carried on a magnetic disk and/or optical disc of a remote computer. A remote computer might load the instructions into its dynamic memory and send the instructions as signals over a transmission medium to be received and/or executed by the computer system 500.

The communications subsystem 512 (and/or components thereof) generally will receive signals, and the bus 502 then might carry the signals (and/or the data, instructions, etc. carried by the signals) to the working memory 514, from which the processor(s) 504 retrieves and executes the instructions. The instructions received by the working memory 514 may optionally be stored on a non-transitory storage device 510 either before or after execution by the processor(s) 504.

It should further be understood that the components of computer device 500 can be distributed across a network. For example, some processing may be performed in one location using a first processor while other processing may be performed by another processor remote from the first processor. Other components of computer system 500 may be similarly distributed. As such, computer device 500 may be interpreted as a distributed computing system that performs processing in multiple locations. In some instances, computer system 500 may be interpreted as a single computing device, such as a distinct laptop, desktop computer, or the like, depending on the context.

The invention has now been described in detail for the purposes of clarity and understanding. However, it will be appreciated that certain changes and modifications may be practiced within the scope of the appended claims.

What is claimed is:

1. A method of detecting a suction event in a blood pump, comprising:
determining at least one operating parameter of a blood pump;
determining, based on the at least one operating parameter of the blood pump, a characteristic waveform signal of the blood pump;
calculating a suction index of the characteristic waveform signal; and
determining when the suction index exceeds a predefined threshold; and
identifying the occurance of a suction event when the suction index exceeds the predefined threshold.

2. The method of detecting a suction event in a blood pump of claim 1, wherein the at least one operating parameter comprises:
pump power;
pump voltage;
pump current;
pump speed.

3. The method of detecting a suction event in a blood pump of claim 1, wherein the characteristic waveform signal comprises:
a representation of flow per time, current per time or power per time of the blood pump.

4. The method of detecting a suction event in a blood pump of claim 1, wherein the suction index is calculated based on a Harmonic Spectral Distribution by executing a Fast Hartley Transform on the characteristic waveform signal.

5. The method of detecting a suction event in a blood pump of claim 4, wherein calculating the Harmonic Spectral Distribution further comprises:
executing a Discrete Bracewell Transform to evaluate the Fast Hartley Transform.

6. The method of detecting a suction event in a blood pump of claim 1, wherein determining whether the suction condition exists comprises:
determining whether the suction index is greater than 20%.

7. The method of detecting a suction event in a blood pump of claim 1, further comprising:
lowering a speed of the blood pump using a binary search method in response to a determination that the suction event has occurred.

8. The method of detecting a suction event in a blood pump of claim 7, wherein lowering the speed of the blood pump comprises:
lowering the speed of the pump to a speed which is based at least in part on the Harmonic Spectral Distribution and a low pump speed limit.

9. The method of detecting a suction event in a blood pump of claim 7, further comprising:
increasing the speed of the blood pump using a binary search method until a set-point speed is restored.

10. The method of detecting a suction event in a blood pump of claim 1, further comprising:
determining if the characteristic waveform signal is valid based at least in part on at least one pump performance characteristic of the blood pump; and
providing a notification when the characteristic waveform signal is not valid.

11. A method of detecting a suction event in a blood pump, comprising:
determining a characteristic waveform signal based on an operating parameter of the blood pump;
calculating a pulse suction index (Ψ) of the waveform signal as Harmonic Spectral Distribution (HSD):
$$Ψ = HSD = 100 \times \frac{( {\sum }_{x = 2}^{x = n} {H}_{x} + {H}_{1 / 2} )}{{H}_{1}}$$
$$Where , \\ \begin{matrix}{H}_{x} = Value of Harmonic x . \\ {=}^{n} Minimum value is 2 and maximum is from 5 to 9.\end{matrix}$$
determining when the pulse suction index exceeds a predefined threshold; and
identifying the occurance of a suction event when the pulse suction index exceeds the predefined threshold.

12. The method of claim 11, wherein HSD is calculated in consecutive time intervals to identify the change in HSD over a period and calculating MEAN value of HSD.

13. The method of claim 11, wherein an increase in MEAN value of HSD indicates a suction event.

14. The method of claim 12, wherein a decrease in MEAN value of HSD with the decrease in speed may additionally indicate recovery from suction condition.

15. The method of claim 11, wherein a lowest allowable pump speed for recovery from suction condition is based on a Low Speed Limit.

16. The method of claim 11, further comprising, decreasing a speed of the pump in response to identification of the existence of a suction condition.

17. A pump system, comprising:
a blood pump; and
a controller that is configured to control the blood pump, the controller configured to:
determine at least one operating parameter of a blood pump;
determine, based on the at least one operating parameter of the blood pump, a characteristic waveform signal of the blood pump;
calculate a suction index of the characteristic waveform signal;
determine when the suction index exceeds a predefined threshold; and
identify the occurance of a suction event when the suction index exceeds the predefined threshold; and
provide an output based on the identification of the suction event.

18. The system of claim 17, wherein the at least one operating parameter comprises:
pump power;
pump voltage;
pump current;
pump speed.

19. The system of claim 17, wherein the characteristic waveform signal comprises:
a representation of flow per time, current per time or power per time of the blood pump.

20. The system of claim 1, wherein calculating the Harmonic Spectral Distribution comprises:
executing a Fast Hartley Transform on the characteristic waveform signal.

21. The system of claim 20, wherein calculating the Harmonic Spectral Distribution further comprises:
executing a Discrete Bracewell Transform to evaluate the Fast Hartley Transform.
